(19) United States
(12) Patent Application Publication
(10) Pub. No.: US 2026/0368916 A1
(43) Pub. Date: Nov. 19, 2026

(54) MULTI-DIMENSIONAL PUPILLARY DISTANCE MAPPING

(71) Applicant: Walmart Apollo, LLC, Bentonville, AR (US)

(72) Inventors: Jacob Cohen, Ashkelon (IL); Neor Ozeri, Ramat Gan (IL); Geva Bidner, Modi'in (IL); Yonatan Blum, Givatayim (IL)

(21) Appl. No.: 19/211,679

(22) Filed: May 19, 2025

(51) Int. Cl. G06T 7/60; A61B 3/00; A61B 3/11; G06V 40/16

(52) U.S. Cl. CPC G06T 7/60; A61B 3/0025; A61B 3/111; G06V 40/165; A61B 2560/0238; G06T 2207/20081; G06T 2207/30041; G06T 2207/30201

(57) ABSTRACT

System and methods for generating accurate pupillary distances. Examples include performing, by a machine learning (ML) model, pixel-to-millimeter conversion for each of a plurality of images of a subject’s face. Examples include measuring, by the ML model, a set of distances for each image, each set of distances comprising a first distance between a first pupil and a second pupil of the face, a second distance between the first pupil and a center of the face, and a third distance between the second pupil and the center of the face. Examples include generating a first distance vector representing the first distance for each image, a second distance vector representing the second distance for each image, and a third distance vector representing the third distance for each image. Examples include generating a multi-dimensional pupillary distance set associated with the remote subject using the distance vectors.

### BACKGROUND

Measuring the pupillary distance (PD) of a subject is a frequently performed step in various ophthalmological and optometric applications. For example, the effectiveness of a subject’s eyeglasses prescription can directly depend on the accuracy of the subject’s PD measurement. Traditionally, PDs are measured by specially trained practitioners, either manually with the use of a ruler, or by employing a specialized piece of medical equipment called a pupilometer, for example.

### SUMMARY

The disclosed examples are described in detail below with reference to the accompanying drawing figures listed below. The following summary is provided to illustrate some examples disclosed herein.

Disclosed are various systems and methods for measuring pupillary distances. Examples include performing, by a machine learning (ML) model, pixel-to-millimeter conversion for each of a plurality of images of a subject’s face. A set of distances is measured for each image by the ML model. Each set of distances includes a first distance between a first pupil and a second pupil of the face, a second distance between the first pupil and a center of the face, and a third distance between the second pupil and the center of the face. A first distance vector representing the first distance for each image, a second distance vector representing the second distance for each image, and a third distance vector representing the third distance is generated for each image. A multi-dimensional pupillary distance set associated with the remote subject is generated using the generated distance vectors.

### BRIEF DESCRIPTION OF THE DRAWINGS

The disclosed examples are described in detail below with reference to the accompanying drawing figures listed below:

FIG. 1 is a block diagram illustrating an example system for determining pupillary distances using a machine learning (ML) model applied to a set of images;

FIG. 2 is a block diagram illustrating another example system for determining pupillary distances using a ML model applied to a set of images;

FIG. 3. is a block diagram illustrating an example ML model measuring pupillary distances from a set of images of a subject’s face;

FIGS. 4A and 4B are diagrams illustrating example measurements taken by a ML model from an image of a subject’s face;

FIG. 4C illustrates example facial features detected by a ML model in performing facial landmark detection.

FIG. 5 is a block diagram illustrating an example of how distance vectors are generated using the pupillary distance measurements described in FIGS. 3, 4A and 4B;

FIG. 6 is a block diagram illustrating an example of how final pupillary distance measurements are determined with the use of a regression model applied to the distance vectors described in FIG. 5; and

FIG. 7 is a flowchart illustrating an example method for determining pupillary distances using a ML model applied to a set of images.

Corresponding reference characters indicate corresponding parts throughout the drawings.

### DETAILED DESCRIPTION

A more detailed understanding can be obtained from the following description, presented by way of example, in conjunction with the accompanying drawings. The entities, connections, arrangements, and the like that are depicted in, and in connection with the various figures, are presented by way of example and not by way of limitation. As such, any and all statements or other indications as to what a particular figure depicts, what a particular element or entity in a particular figure is or has, and any and all similar statements, that can in isolation and out of context be read as absolute and therefore limiting, can only properly be read as being constructively preceded by a clause such as “In at least some embodiments, ...” For brevity and clarity of presentation, this implied leading clause is not repeated ad nauseum.

Pupillary Distance (PD) measurements are a frequently performed step in various ophthalmological and optometric applications. Traditionally, PDs are measured by specially trained practitioners, either manually with the use of a ruler, or by employing a specialized piece of medical equipment called a pupilometer. These methods rely on the physical presence of the subject at a location of the trained practitioner. Manual measurements are often inaccurate, and only specially trained practitioners are generally able to use pupilometers effectively. Accordingly, conventional PD measurements have been known to be cumbersome, time-consuming, inaccessible and/or inaccurate.

Aspects of the disclosure solve multiple problems that are necessarily rooted in computer technology, and render use of computing platforms more efficient by providing improved systems and methods for determining PDs using computer vision technology and techniques. Specifically, some examples include a computing device that receives a plurality of images of a face of a remote subject from an image capture device and performs a pixel-to-millimeter conversion for each image in the plurality of images. Examples include the computing device measuring, by a machine learning (ML) model, a set of distances for each image in the plurality of images using a result of the pixel-to-millimeter conversion for each image, each set of distances comprising a first distance between a first pupil and a second pupil of the face, a second distance between the first pupil and a center of the face, and a third distance between the second pupil and the center of the face. Examples include the computing device generating a first distance vector representing the first distance for each image of the plurality of images, a second distance vector representing the second distance for each image of the plurality of images, and a third distance vector representing the third distance for each image of the plurality of images. Examples include the computing device generating a multi-dimensional pupillary distance set associated with the first pupil and the second pupil of the remote subject using the first distance vector, the second distance vector, and the third distance vector, wherein accurate corrective optical fitting is enabled for the remote subject using the multi-dimensional pupillary distance set.

According to various embodiments, the computing device recognizes facial features from the captured images and performs pixel-to millimeter conversion based on facial landmarking techniques. This feature recognition within the image provides for efficient use of in-image features in performing pixel-to-millimeter conversions.

According to various embodiments, the ML model and/or regression model is spread across a plurality of servers of a server pool, thereby providing increased efficiency of processing power and time in processing the plurality of images.

According to various embodiments, the computing device calculates pupillary distances from a plurality of images, thereby increasing accuracy of the output PD calculations provided by the computing device.

According to various embodiments, the computing device delivers final calculated pupillary distances calculated by the regression model to the ML model as training data, thereby improving accuracy in future distance measurements measured by the ML model on input images.

According to various embodiments, the multi-dimensional pupillary distance set comprises a final first distance, a final second distance, and a final third distance. Thereby, the computing device is able to provide multiple pupillary distance measurements enabling various applications related to corrective optical fittings.

According to various embodiments, the ML model generates a comprehensive three-dimensional geometrical structure of the face using multi-dimensional analysis of the face, thereby providing for highly accurate measurements in measuring the first, second, and third distances.

The various examples will be described in detail with reference to the accompanying drawings. Wherever preferable, the same reference numbers will be used throughout the drawings to refer to the same or like parts. References made throughout this disclosure relating to specific examples and implementations are provided solely for illustrative purposes but, unless indicated to the contrary, are not meant to limit all examples.

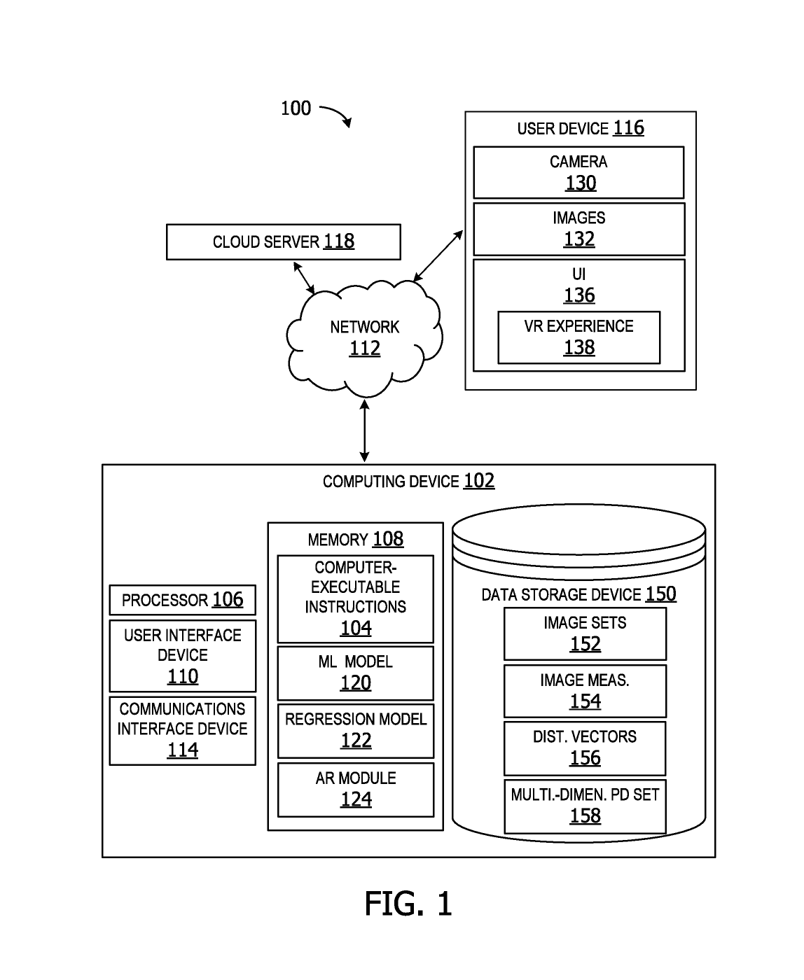

FIG. 1 illustrates an example block diagram of a system 100 for measuring pupillary distances (PD). Pupillary distances can comprise various distances measured related to the eyes of a subject. Pupillary distances can include a distance between the center of the left pupil to the center of the right pupil. In some embodiments, pupillary distances can include the distance from the center of the right pupil to a centerline of the subject’s face, and the distance from the center of the left pupil to the centerline of the subject’s face. In the example of FIG. 1, computing device 102 represents any device executing computer-executable instructions 104 (e.g., as application programs, operating system functionality, or both) to implement the operations and functionality associated with the computing device 102. Computing device 102 can represent a group of processing units or other computing devices. In some embodiments, the computing device 102 has at least one processor 106 and a memory 108. Computing device 102, in other embodiments includes a user interface device 110.

The processor 106 includes any quantity of processing units (such as a microcontroller, a microprocessor, central processing unit core(s), an ASIC, an FPGA, and/or other similar hardware device) and is suitable to execute the computer-executable instructions 104. The computer-executable instructions 104 are performed by the processor 106, performed by multiple processors within the computing device 102, or performed by a processor external to the computing device 102. In some embodiments, processor 106 is programmed to execute instructions such as those illustrated in the figures.

The computing device 102 further has one or more computer-readable media such as the memory 108. The memory 108 includes any quantity of media associated with or accessible by the computing device 102. The memory 108 in these examples is internal to computing device 102 (as shown in FIG. 1). In other embodiments, the memory 108 is external to the computing device (not shown) or both (not shown). The memory 108 can include a read-only memory and/or memory wired into an analog computing device.

The memory 108 stores data, such as one or more applications, including machine learning (ML) model 120 configured to utilize computer vision and machine learning techniques to identify and/or recognize objects from images and/or measure distances between recognized objects of the images, such as images from user device 116, for example; a regression model 122 trained to provide PD information based on measurement vectors representing various measurements measured by ML model 120 from multiple images; and an augmented reality (AR) module 124 configured to generate AR experiences for gathering images of a subject’s pupils via user device 116. In some embodiments, ML model 120 is an image recognition as a service (IRAS) deep learning model trained to detect and recognize an object of interest in one or more images, and can be trained using labeled training data. The applications, when executed by processor 106, operate to perform functionality on the computing device 102. The applications can communicate with counterpart applications or services such as web services accessible via a network 112 or multiple communication networks 112. In an example, the applications represent downloaded client-side applications that correspond to server-side services executing in a cloud.

In other embodiments, the user interface device 110 includes a graphics card for displaying data to the user and receiving data from the user. The user interface device 110 can also include computer-executable instructions (e.g., a driver) for operating the graphics card. Further, the user interface device 110 can include a display (e.g., a touch screen display or natural user interface) and/or computer-executable instructions (e.g., a driver) for operating the display. The user interface device 110 can also include one or more of the following to provide data to the user or receive data from the user: speakers, a sound card, a camera, a microphone, a vibration motor, one or more accelerometers, a BLUETOOTH® brand communication module, wireless broadband communication (LTE) module, global positioning system (GPS) hardware, and a photoreceptive light sensor. In a non-limiting example, the user inputs commands or manipulates data by moving the computing device 102 in one or more ways.

Network 112 is implemented by one or more physical network components, such as, but without limitation, routers, switches, network interface cards (NICs), and other network devices. Network 112 is any type of network for enabling communications with remote computing devices, such as, but not limited to, a local area network (LAN), a subnet, a wide area network (WAN), a wireless (Wi-Fi) network, or any other type of network. In this example, network 112 is a WAN, such as the Internet. However, in other embodiments, network 112 is a local or private LAN.

In some embodiments, computing device 102 optionally includes a communications interface device 114. The communications interface device 114 includes a network interface card and/or computer-executable instructions (e.g., a driver) for operating the network interface card. Communication between the computing device 102 and other devices, such as but not limited to user device 116, can occur using any protocol or mechanism over any wired or wireless connection. In some embodiments, the communications interface device 114 is operable with short range communication technologies such as by using near-field communication (NFC) tags.

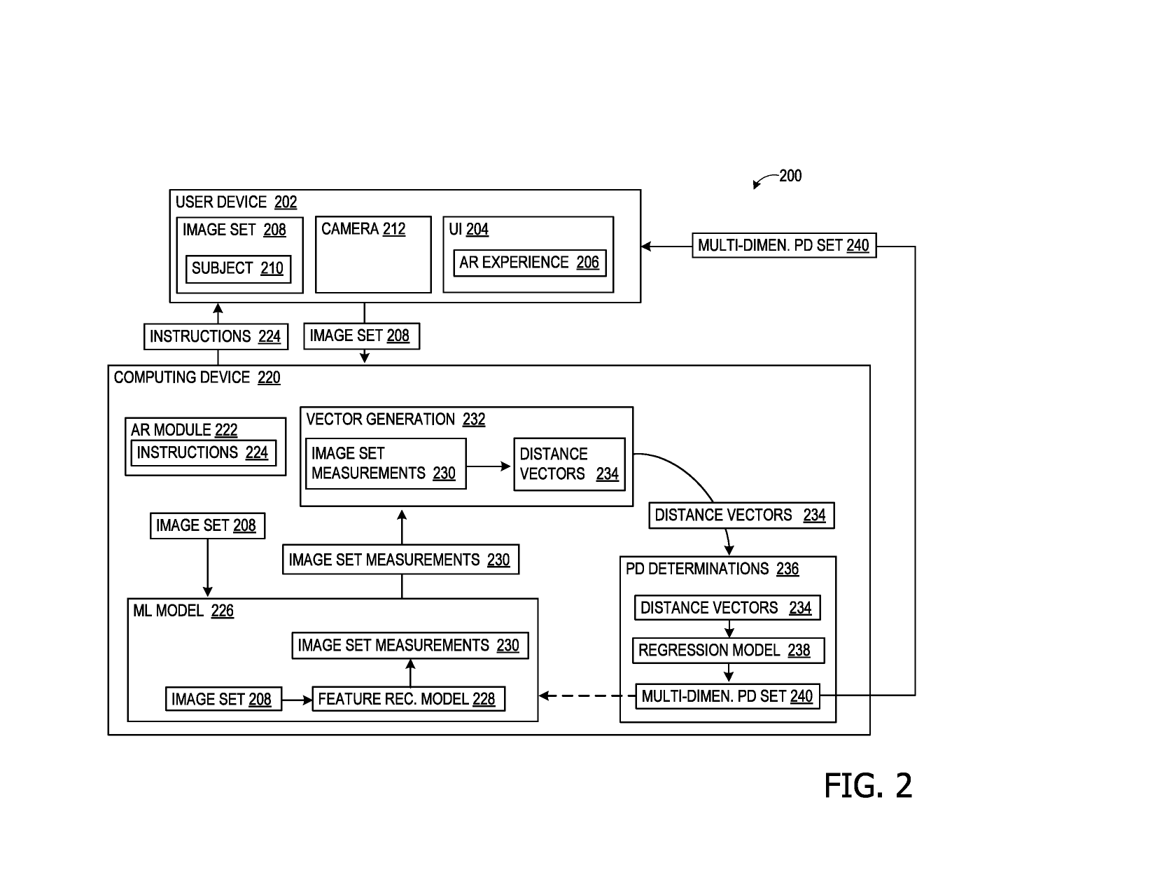

The user device 116 represents any device executing computer-executable instructions. User device 116 can be implemented as a mobile computing device, such as, but not limited to, a wearable computing device, a mobile telephone, laptop, tablet, computing pad, netbook, gaming device, and/or any other portable device. The user device 116 includes at least one processor and a memory. The user device can include an image capture device 130 configured to gather image data used by computing device 102. Image capture device 130 can include a digital camera for capturing images, such as, but not limited to, camera 212 discussed in FIG. 2 below. Image capture device 130 can store and/or capture images 132 utilized by computing device 102 for determining PDs. User device 116 includes a user interface 136, which may be substantially the same as user interface device 110 previously discussed. AR module 124 can display to the user an interactive AR experience 138 including instructions for generating images 132 capturing the subject’s face via user interface 136.

The cloud server 118 is a logical server providing services to the computing device 102 or other clients, such as, but not limited to, the user device 116 and computing device 102. Cloud server 118 is hosted and/or delivered via network 112. In some non-limiting examples, cloud server 118 is associated with one or more physical servers in one or more data centers. In other embodiments, the cloud server 118 is associated with a distributed network of servers. In some embodiments, cloud server 118 provides additional computing service and/or processing power for applications of computing device 102, such as providing additional processing power for ML model 120 in processing multiple images 132 simultaneously.

The system 100 can optionally include a data storage device 150 for storing data, such as, but not limited to, image sets 152 comprising a plurality of the images 132 from user device 116; image measurements 154 measured from the images sets 152 by ML model 120; distance vectors 156 generated by computing device 102 based on images measurements 154; and multi-dimensional PD sets 158 generated by regression model 122 based on distance vectors 156. The data storage device 150 can include one or more different types of data storage devices, such as, for example, one or more rotating disks drives, one or more solid state drives (SSDs), and/or any other type of data storage device. The data storage device 150 in some non-limiting examples includes a redundant array of independent disks (RAID) array. In some non-limiting examples, the data storage device(s) provide a shared data store accessible by two or more hosts in a cluster. For example, the data storage device can include a hard disk, a redundant array of independent disks (RAID), a flash memory drive, a storage area network (SAN), or other data storage device. In other embodiments, the data storage device 150 includes a database.

The data storage device 150 in this example is included within the computing device 102, attached to the computing device, plugged into the computing device, or otherwise associated with the computing device 102. In other embodiments, data storage device 150 includes a remote data storage accessed by the computing device via network 112, such as a remote data storage device, a data storage in a remote data center, or a cloud storage. Similarly, ML model 120, regression model 122, and AR module 124 are depicted within computing device 102, according to various embodiments, these applications can be implemented on a remote or cloud memory storage.

FIG. 2 is a block diagram illustrating an example system 200 for determining pupillary distances (PD). Various elements of system 200 correspond with elements of system 100 previously discussed. System 200 includes a user device 202 accessed by a subject 210 for measuring PDs of subject 210. User device 202 includes a user interface (UI) 204 that displays an augmented reality (AR) experience 206 for guiding subject 210 through the process of generating PD measurements. AR experiences, such as AR experience 206, combine real world elements captured by a camera or image capture device with virtual and/or instructional elements. Specifically, the AR experience 206 provides instructions for capturing an image set 208 of a plurality of images containing the subject’s 210 face via camera 212. Image set 208 is then delivered to a computing device 220.

As shown, computing device 220 includes AR module 222 that generates 224 instructions that are received by user device 202 and used as part of AR experience 206. For example, instructions 224 can specify a desired distance or location of subject 210 relative to camera 212 to ensure images of image set 208 are captured with subject 210 positioned at a proper location and/or orientation for computing device 220 to perform PD measurements. AR module 222 can analyze live streaming data from camera 212 and provide instructions accordingly. For example, in some embodiments, the instructions 224 instruct subject 210 to move toward/away camera 212, or to change the orientation of their head based on a detected location of subject 210 within the streaming data from camera 212. In some embodiments, AR module 222 automatically captures the images of image set 208 in response to determining that subject 210 is in the proper position and at the proper orientation, for example. Thus, instructions 224 are guided instructions for calibrating parameters of the subject’s 210 face relative to camera 212 to improve image quality of the plurality of images of image set 208 captured by the image camera 212. While instructions 224 are described herein as being part of an AR experience 206, those with skill in the art will understand that various embodiments fall within the scope of this disclosure. For example, in some embodiments, instructions 224 simply direct the subject 210 or a practitioner in how to position subject 210 relative to camera 212.

Computing device 220 measures various distances associated with the images of image set 208 using a machine learning (ML) model 226. Specifically, in some embodiments, ML model 226 utilizes a recognition model 228, which can be a computer vision model, such as a deep learning model, trained to recognize and/or identify various features related to the images of image set 208, such as pupils and other facial features of subject 210 using any of various computer vision applications and techniques. In some examples, ML model 226 comprises a regression model, such as regression model 238. Accordingly, ML model 226 utilizes machine learning, and specifically computer vision, techniques in identifying features and generating measurements from image set 208. In some examples, recognition model 228 and/or ML model 226 comprises a convolutional neural network (CNN) model. With the identified facial features and pupils of subject 210, ML model 226 performs pixel-millimeter conversions to determine various measurements associated with the pupils of subject 210. ML model 226 can employ depth perception, lighting, shading, and facial landmarking techniques in measuring pixel distances of the various images of image set 208 and performing pixel-to-millimeter conversions. Thereby, ML model 226 generates image set measurements 230 related to the images of image set 208. As will be discussed in greater detail below, image set measurements 230 include a plurality of distance measurements from each image of image set 208.

Computing device 220 performs vector generation 232 using image set measurements 230. As will be discussed in greater detail below, computing device 220 generates distance vectors 234, which includes a distance vector corresponding to each unique distance-type measured in image set measurements 230. For example, distance vectors 234 can include a distance vector for pupillary distances between the subject’s two pupils measured in image set 208, a distance vector for right pupil to face mid-line distances measured in image set 208, and a distance vector for left pupil to face mid-line distances measured in image set 208, as will be discussed in greater detail below. Each distance vector in distance vector 234 represents numerous data points taken across various images, and thus each vector generated during vector generation 232 is used to store various numerical data points associated with a certain measurement relating to the subject’s 210 pupils.

Using distance vectors 234, computing device 220 performs PD determinations 236. Specifically, computing device 220 applies a regression model 238 to each of the vectors in distance vectors 234 to determine a multi-dimensional PD set 240. Specifically, multi-dimensional PD set 240 includes a final measurement corresponding to each of the vectors included in distance vectors 234. Regression model 228 comprise machine learning and/or deep learning trained to determine various pieces of PD information using various inputs, such as distance vectors 234 and/or image set 208. For example, according to some embodiments, recognition model 228 comprises a convolutional neural network (CNN). In some embodiments, regression model 238 is included as part of ML model 226.

Multi-dimensional PD set 240 can then be delivered to a user device, such as user device 202 for example. Multi-dimensional PD set 240 can be utilized in various ophthalmological and optometric applications, such as in application related to corrective prescription eyewear, for example. Accordingly, in some embodiments, user device 202 or another device receiving multi-dimensional PD set 240 is a device belonging to an eye health professional in providing ophthalmological or optometric care to a patient. In some embodiments, user device 202 or another device receiving multi-dimensional PD set 240 is a device belonging to an individual, such as an individual using user device 202 in an e-commerce setting for purchasing new prescription eyewear, or in providing pupillary distance information to an eye care professional as part of a virtual appointment, for example. Thus, in some embodiments, subject 210 can be described as a remote subject that is remote or displaced from any eyecare facility or technician.

Additionally, in some embodiments, multi-dimensional PD set 240 computing device 220 uses multi-dimensional PD set 240 as training data for ML model 226 to thereby improve accuracy of future measurements taken from images processed by ML model 226.

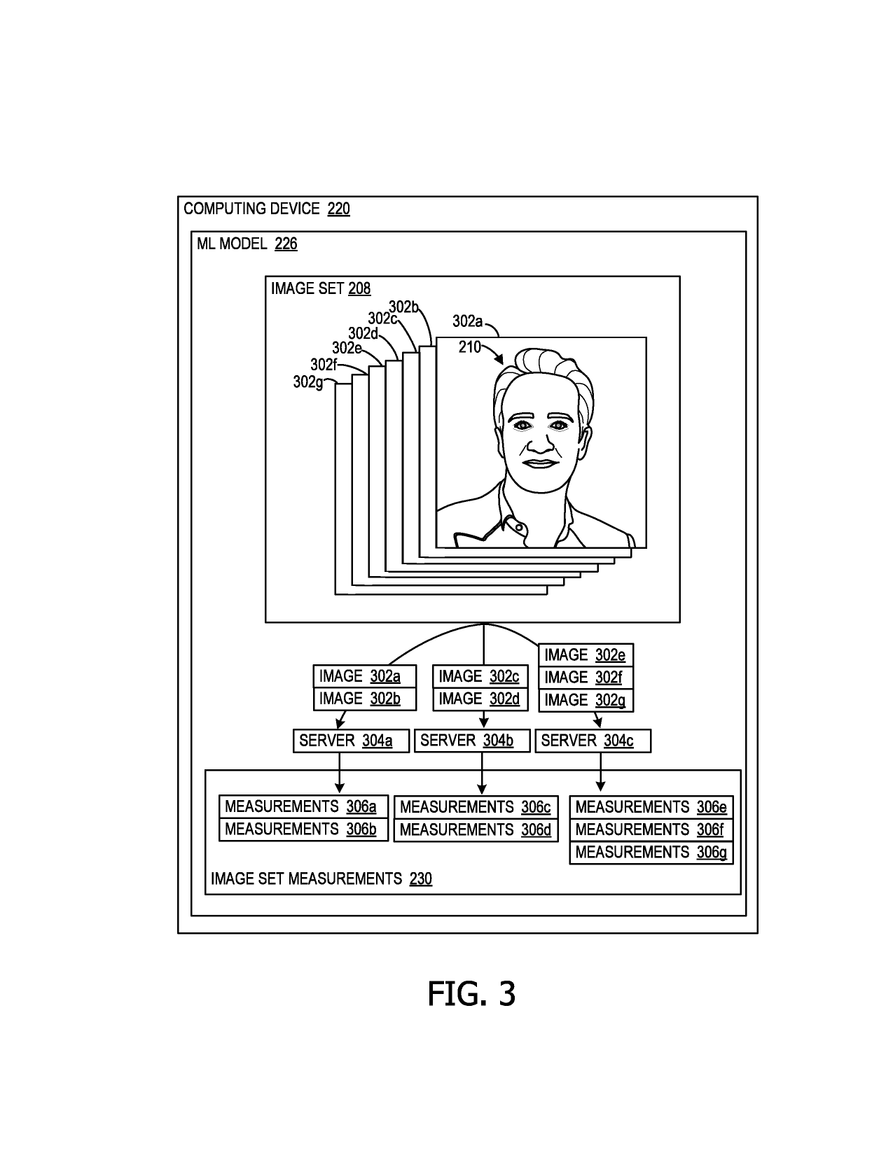

FIG. 3 is a block diagram illustrating an example of ML model 226 generating image set measurements 230 from image set 208. As previously discussed, image set 208 includes a plurality of images 302 containing subject 210. As shown, in this illustrative example, image set 208 includes seven images 302a-302g. As those with skill in the art will understand, seven images 302a-302g is used herein as an illustrative example, and image set 208 can include more or fewer than seven images 302 according to various embodiments of this disclosure.

According to some embodiments, computing device 220 controls camera 212 to capture a predetermined number of images 302 in rapid succession upon activation by a user via UI 204, or by computing device 220 determining from streaming data from camera 212 that subject 210 is positioned relative to camera 212 at a desired distance and/or orientation. In some embodiments, a user of user device 202 captures each of the plurality of images 302 individually using a camera interface, such as via UI 204 or an interface associated with camera 212, for example. Determining PD measurements of subject 210 using an image set 208 comprising a plurality of images 302 provides for better accuracy in providing final PD measurements compared to only using one image. Each of the plurality of images 302 will contain minor differences than the other of the images, providing unique features and perspectives for ML model 226 to use in generating image set measurements 230. For example, the subject’s 210 facial orientation or distance relative to camera 212 may differ slightly across the plurality of images 302, thereby providing a fuller and more complete understanding of the facial geometry for ML model 226. Additionally, the subject’s 210 eye direction or focus location for subject’s 210 eyes may change across the plurality of images 302, thereby providing ML model 226 with a more complete understanding of subject’s pupils locations.

ML model 226 determines various measurements associated with each image 302 in generating image set measurements 230. In some embodiments, ML model 226 is implemented on a plurality of servers 304 forming a server pool, and the images are spread across the servers 304 of the server pool for processing. As shown, in this illustrative example, there are three servers 304a-304c. The server pool can comprise more or fewer than three servers 304 according to various embodiments of this disclosure. Spreading processing of images 302 across numerous servers 304 of the server pool improves efficiency of and processing time of ML model 226. However, according to some embodiment, ML model 226 is implemented on a single server 304 rather than a pool of servers.

ML model 226 may spread images 302 across the servers 304 of the server pool. As shown, in this illustrative example, images 302a-302b are delivered to server 304a; images 302c-302d are delivered to server 304b; and images 302e-302g are delivered to server 304a. Each server 304 generates measurements 306 related to the images 302 processed by the server 304. As shown, server 304a generates measurements 306a corresponding to image 302a and measurements 306b corresponding to image 302b; server 304b generates measurements 306c corresponding to image 302c and measurements 306d corresponding to image 302d; and server 304c generates measurements 306e corresponding to image 302e, measurements 306f corresponding to image 302f, and measurements 306g corresponding to image 302g.

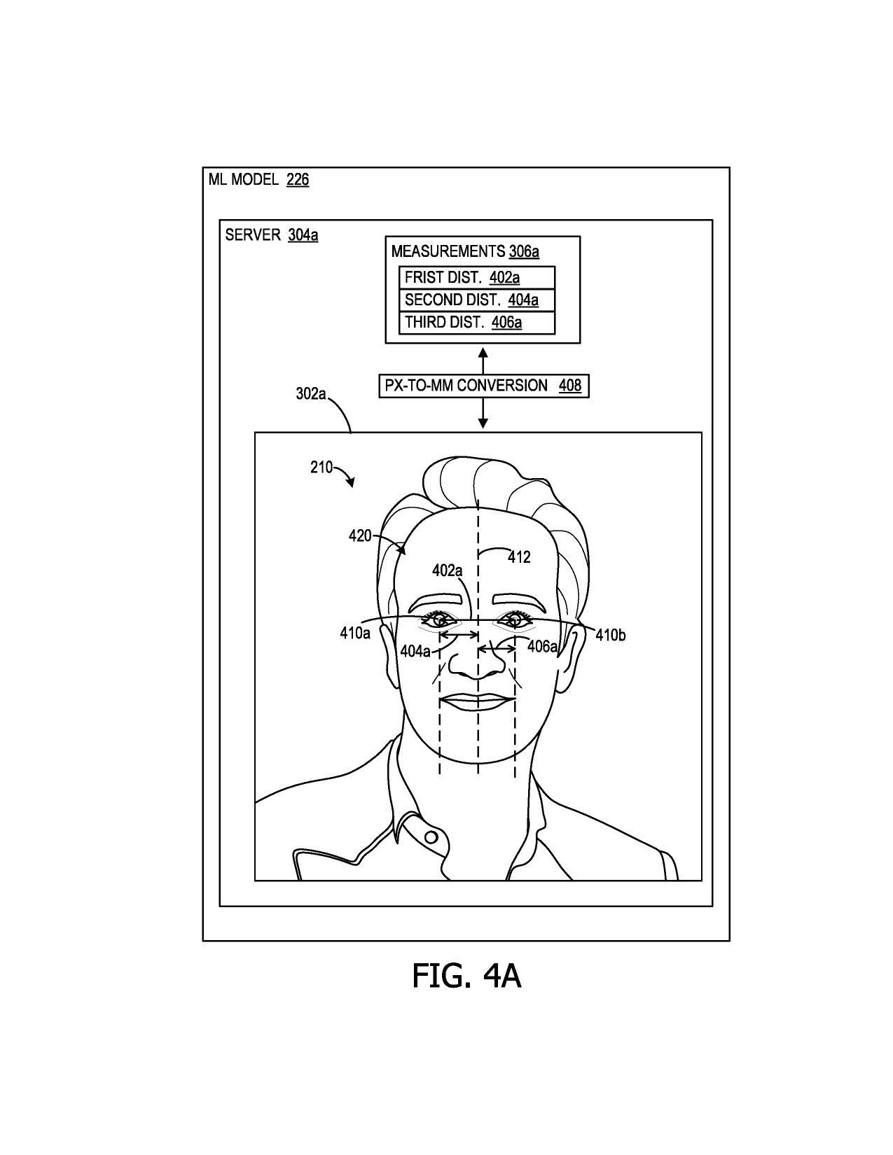

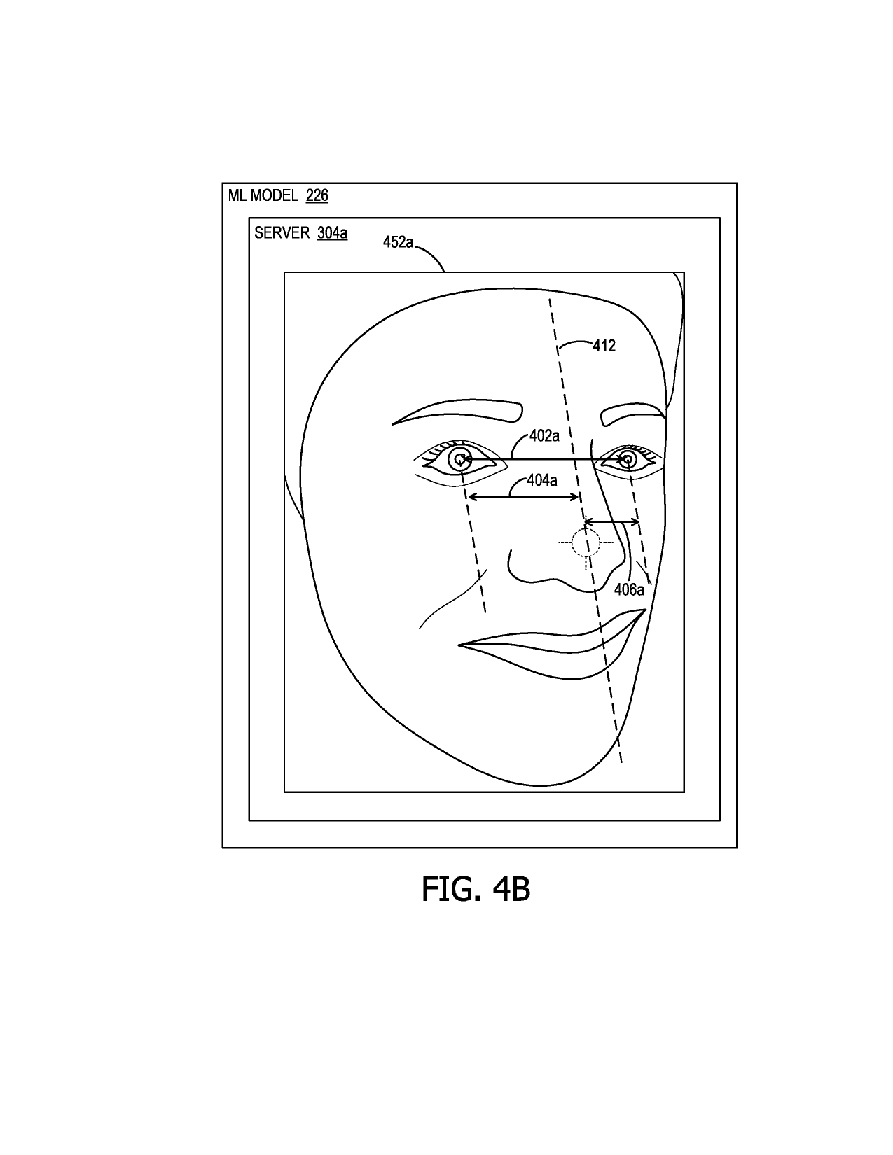

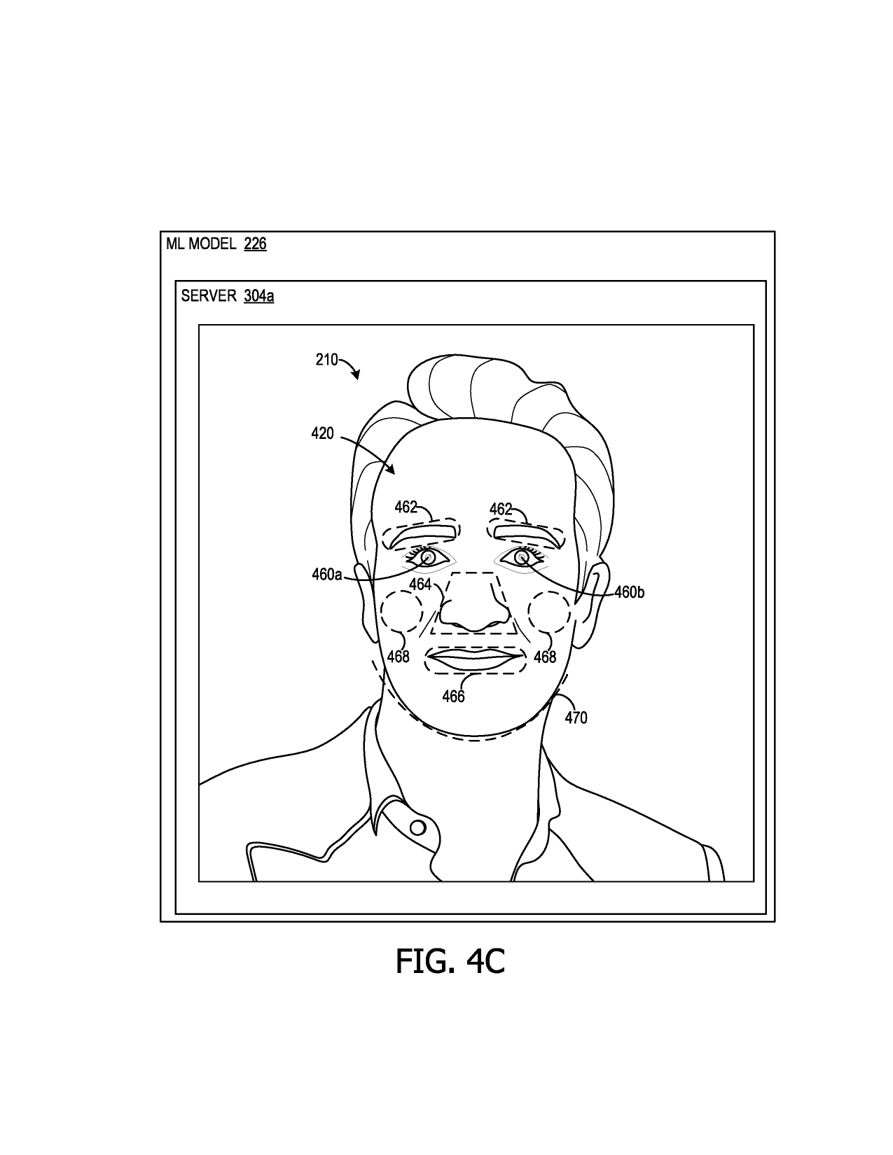

FIG. 4A illustrates various distances measured from images 302 and included in measurements 306 by ML model 226. FIG. 4A is an illustrative example utilizing image 302a and server 304a, but those with skill in the ear will understand that FIG. 4A discussion is applied to each of the images 302 and servers 304 in forming each of the measurements 306, and that image 302a is simply used in FIG. 4A for illustrative purposes in describing the measurements included in measurements 306.

Measurements 306 include numerous distance measurements taken from each image 302, including a first distance 402, a second distance 404, and a third distance 406. Since FIG. 4A uses image 302a for an illustrative example, each of the distance measured from image 302a and included in measurements 306a is signified with an “a” qualifier; i.e. first distance 402a, second distance 404a, and third distance 406a. Those with skill in the art will understand that analogous naming conventions will be used for the other illustrative images 302b-302g and associated measurements 306b-306g.

ML model 226 and/or recognition model 228 performs depth perception, lighting, shading, and facial landmarking techniques on face 420 of subject 210. By such techniques, the facial geometry and landmarks of face 420 are identified, and a centerline 412 of face 420 is determined by ML model 226. Additionally, as previously discussed, ML model 226 and/or recognition model 228 is trained in recognizing and/or segmenting subject’s 210 pupils 410 using pupil centering identification. As shown, ML model 226 identifies subject’s right pupil 410a and left pupil 410b.

Using the identified facial features and landmarks, ML model 226 calculates pixel distances for first distance 402a, second distance 404a, and third distance 406a from image 302a. ML model 226 performs pixel-to-millimeter conversion 408 using the measured pixel distance to convert the measured pixel distances to a standard unit of measure, such as millimeters, for example. Pixel-to-millimeter conversion 408 comprises converting pixels of an image to a unit of measure, such as millimeters, so that features of the images that are represented in pixels can be assessed and measured in millimeters. In some embodiments, ML model 226 performs depth perception, lighting, shading, and facial landmarking techniques in determining pixel-to-millimeter conversion factors in performing pixel-to-millimeter conversion. Additionally, in some examples, ML model 226 utilizes predetermined or instructed data related to subject’s 210 known position relative to camera 212 when image 302a was captured by camera 212. For example, the distance between subject 210 and camera 212 may be a known distance, such as a distance specified in instructions 224, and this distance between subject 210 and camera 212 can be utilized by ML model 226 in generating conversion factors for performing pixel-to-millimeter conversion 408. Those with skill in the art will understand that while converting pixel distance measurements to millimeters is described herein, various other embodiments of this disclosure include converting pixel distance measurements to any of various other standard units of measure.

Referring to FIG. 4B, in some embodiments, ML model 226 further performs three-dimensional modeling of face 420 in generating measurements 306a. Using computer vision and segmentation techniques, such as the various techniques discussed herein, ML model 226 generates a comprehensive 3D geometrical structure 452a of 420. From 3D geometrical structure 452a, ML model 226 can generate highly accurate measurements for first distance 402a, second distance 404a, and third distance 406a,

FIG. 4C illustrates various facial landmarks of face 420 that can be identified and used by ML model 226 in performing landmark detection. ML model 226 detects and generates pupil center coordinates 460 corresponding to the center of the pupils 410. As shown, there is a right pupil center coordinate 460a and a left pupil center coordinate 460a. These pupil center coordinates 460 are used by ML model 226 as landmarks for mapping the structure of face 420 and are used in determining measurements 306.

ML model 226 can further detect various other facial landmarks, such as eyebrows 462, nose 464, mouth 466, cheeks 468, and jawline 470, for example. ML model 226 uses these detected facial landmarks in performing pixel-to-millimeter conversion 408, determining measurements 306, generating facial centerline 412, and generating 3D geometrical structure 452, as has been previously discussed. Those with skill in the art will recognize that the facial landmarks depicted in FIG. 4C are examples of facial landmarks, and that ML model 226 can detect and utilize various other facial landmarks associated with face 420 in performing the various image processing discussed herein.

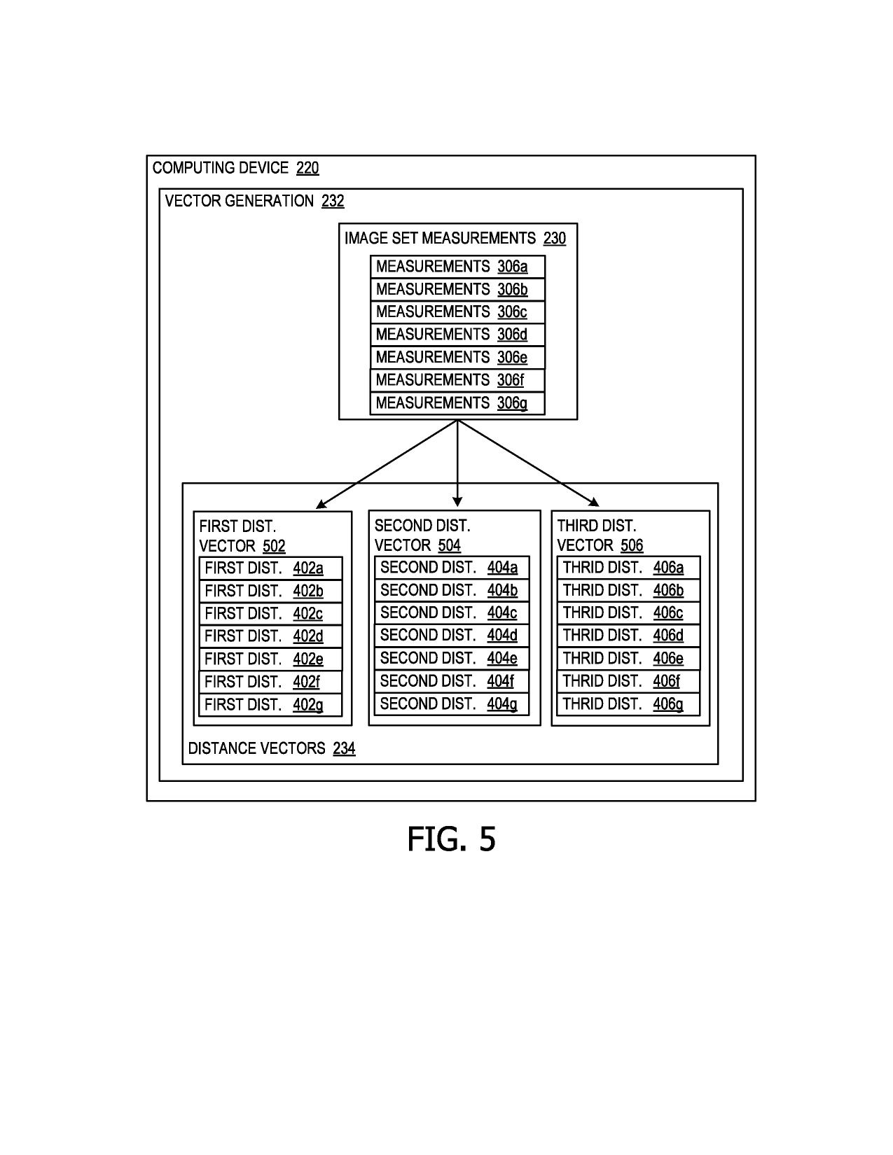

FIG. 5 illustrates an example of computing device 220 performing vector generation 232 to generate distance vectors 234 from image set measurements 230. Specifically, as shown computing device 220 generates a vector corresponding to each unique distance measured within measurements 306. As shown, computing device 220 generates a first distance vector 502 corresponding to first distance 402; a second distance vector 504 corresponding to second distance 404; and a third distance vector 506 corresponding to third distance 406. Each distance vector is comprised of its corresponding distances measurement from each of the measurements 306. As shown, first distance vector 502 includes first distance 402a from measurements 306a, first distance 402b from measurements 306b, and so on. Similarly, second distance vector 504 includes second distance 404a from measurements 306a, second distance 404b from measurements 306b, and so on. Similarly, third distance vector 502 includes third distance 406a from measurements 306a, third distance 406b from measurements 306b, and so on.

Accordingly, first distance vector 502 can be described as including each measured interpupillary distance (first distance 402) from each image 302 of image set 208; second distance vector 504 can be described as including each measured distance from right pupil 410a to facial centerline 412 (second distance 404) from each image 302 of image set 208; and third distance vector 506 can be described as including each measured distance from left pupil 410b to facial centerline 412 (third distance 40) from each image 302 of image set 208.

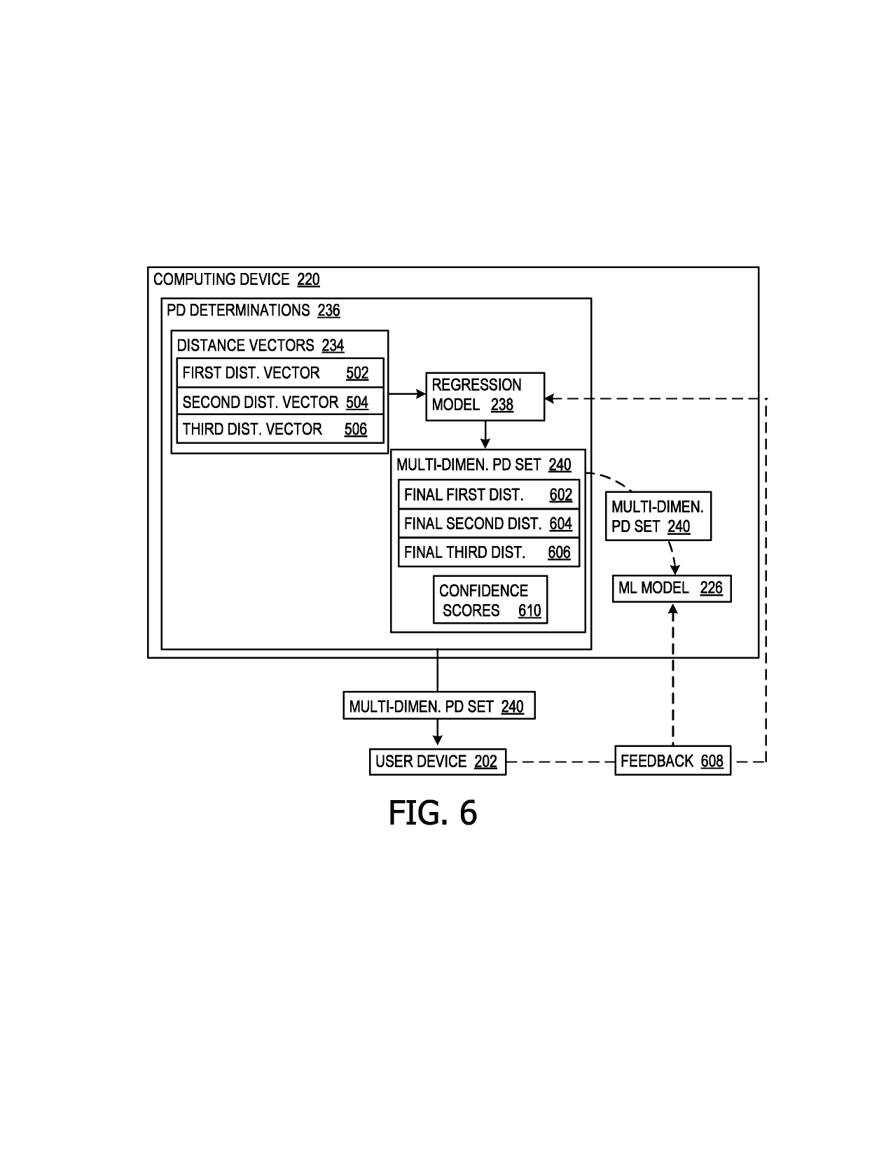

FIG. 6 illustrates an example of computing device 220 performing PD determinations 236 to generate multi-dimensional PD set 240. Computing device 220 applies a regression model to first distance vector 502, second distance vector 504, and third distance vector 506 to thereby generate a final first distance 602, a final second distance 604, and a final third distance 606. Final first distance 602 is generated from first distances 402a-402g of first distance vector 502; final second distance 604 is generated from second distances 404a-404g of second distance vector 504; and final third distance 606 is generated from third distances 406a-406g.

Accordingly, final first distance 602 is a calculated interpupillary distance between pupil 410a and pupil 410b; final second distance 604 is a calculated distance between right pupil 410a and facial centerline 412; and final third distance 606 is a calculated distance between left pupil 410b and facial centerline 412. Regression model 228 is trained to calculate each of the final first distance 602, final second distance 604, and final third distance 606 based on the corresponding input distance vector 502, 504, 506. Thus, by utilizing distance vectors 502, 504, 506 representing measurements 306 taken from a plurality of images 302, regression model 228 is trained to calculate precise and accurate final distance measurements 602, 604, 606 representative of the individual measurements 306 provided as an input.

In some embodiments, multi-dimensional PD set 240 further includes confidence scores 610 related to final first distance 602, final second distance 604, and final third distance 606. The confidence scores 610 indicate the probability that the output of regression model 238 is correct. That is, confidence scores 610 indicate – for each of the final first distance 602, final second distance 604, and final third distance 606 calculated distance – a probability that the calculated distance is the same as the actual corresponding measured value for subject 210. According to various embodiments, the systems herein have been shown to produce confidence scores 610 of 95% and better, thus providing for highly accurate PD calculations.

In some embodiments, computing device 220 delivers multi-dimensional PD set 240 to a user device, such as user device 202. As previously discussed, multi-dimensional PD set 240 can be utilized by an eye health provider operating user device 202 in performing various ophthalmological and optometric applications, such as in providing corrective optical eyewear prescriptions, for example. In some embodiments, user device 202 or another device receiving multi-dimensional PD set 240 is a device belonging to an individual, such as an individual using user device 202 in an e-commerce setting for purchasing new prescription eyewear, or in providing pupillary distance information to an eye care professional as part of a virtual appointment, for example.

In some embodiments, multi-dimensional PD set 240 is provided to ML model 226 as training data for use in performing future measurements. Thereby, ML model 226 uses multi-dimensional PD set 240 to improve accuracy of measurements taken from future images by ML model 226. ML model 226 can analyze measurements 306 taken across the various images 302 relative to the calculated final distances 602, 604, 606 to improve model accuracy in future PD measurements taken from input images. In some embodiments, feedback 608 can be provided to ML model 226 and/or regression model 238 as training data. For example, in some embodiments, feedback 608 is input by a user via user device 202 and includes manual PD measurements corresponding to final distances 602, 604, 606, and such feedback is used by ML model 226 and regression model 238 to improve model accuracy in future PD measurement applications or future distance vector analysis.

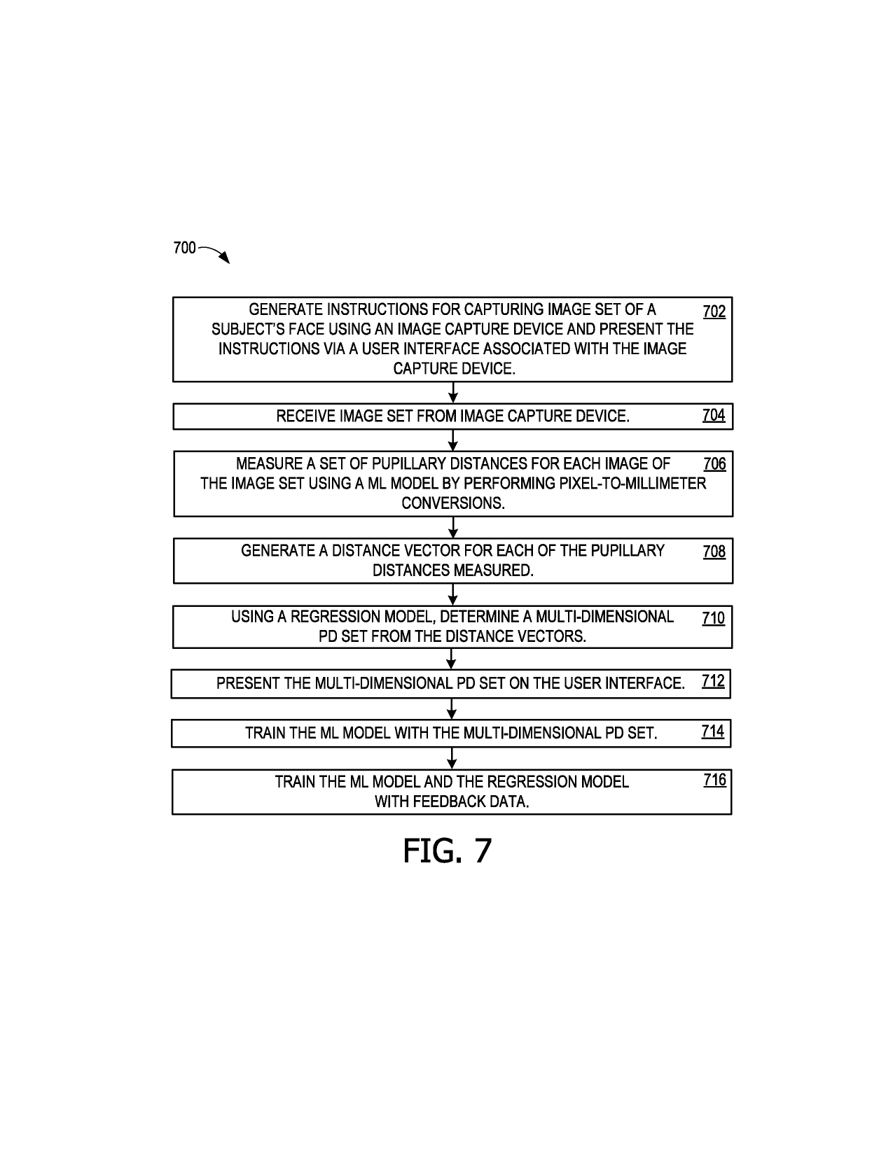

FIG. 7 is a flowchart illustrating an example method 700 for determining pupillary distances using a ML model applied to a set of images. Method 700 can start at block 702 where AR module 222 generates instructions 224 displayed or communicated via UI 204 of user device 202 for capturing image set 208 of subject 210 using camera 212. Instructions 224 are guided instructions for calibrating parameters of the subject’s 210 face relative to camera 212 to improve image quality of the plurality of images 302 of image set 208 captured by the image camera 212, as previously discussed. Method 700 can continue to block 704 where computing device 220 receives image set 208 including the plurality of images 302 from user device 202. The images of the user’s face are provide by the user with the user’s consent.

Method 700 can continue to block 706 where computing device 220, via ML model 226, performs pixel-to-millimeter conversions 408 and thereby determines measurements 306 from each image 302 of image set 208. Method 700 can continue to block 708 where computing device 220 performs vector generation 232 to generate first distance vector 502, second distance vector 504, and third distance vector 506 from measurements 306 taken from each image 302.

Method 700, in some examples, continues to block 710 where computing device 220, using regression model 238, generates multi-dimensional PD set 240 based on first distance vector 502, second distance vector 504, and third distance vector 506. Method 700 can continue to block 712 where computing device 220 delivers multi-dimensional PD set 240 to a user device, such as user device 202, for presenting via user device 202.

Method 700 can optionally continue to block 714 where computing device 220 uses multi-dimensional PD set 240 as training data for training or re-training and thereby finetune and improve the accuracy of ML model 226. Method 700 can optionally continue to block 716 where feedback 608 is used to train or re-train ML model 226 and/or regression model 238. For example, in some embodiments, feedback 608 is input by a user via user device 202 and includes manual PD measurements corresponding to final distances 602-604, and such feedback is used by ML model 226 and regression model 238 to finetune and improve model accuracy in future PD measurement applications or future distance vector analysis.

While method 700 is described as performing block 702-716 in a certain order, those with skill in the art will understand that block 702-716 are performed in various orders according to various embodiments of this disclosure. Additionally, those with skill in the art will recognize that, according to various embodiments of this disclosure, method 700 includes more or fewer than the block 702-716 depicted.

### Additional Examples

In an example scenario, the systems herein (such as system 100 and 200, for example) are utilized by ophthalmological and/or optometric providers, such as for providing eyecare care and/or corrective lenses consultations to their patients. In an example scenario, the systems herein (such as system 100 and 200, for example) are utilized in an e-commerce setting for purchasing new prescription eyewear, or in providing pupillary distance information to an eye care professional as part of a virtual appointment, for example.

Specifically, some examples include a computing device that receives a plurality of images of a face of a remote subject from an image capture device and performs a pixel-to-millimeter conversion for each image in the plurality of images. Examples include the computing device measuring, by a ML model, a set of distances for each image in the plurality of images using a result of the pixel-to-millimeter conversion for each image, each set of distances comprising a first distance between a first pupil and a second pupil of the face, a second distance between the first pupil and a center of the face, and a third distance between the second pupil and the center of the face. Examples include the computing device generating a first distance vector representing the first distance for each image of the plurality of images, a second distance vector representing the second distance for each image of the plurality of images, and a third distance vector representing the third distance for each image of the plurality of images. Examples include the computing device generating a multi-dimensional pupillary distance set associated with the first pupil and the second pupil of the remote subject using the first distance vector, the second distance vector, and the third distance vector, wherein accurate corrective optical fitting is enabled for the remote subject using the multi-dimensional pupillary distance set.

Alternatively, or in addition to the other examples described herein, examples include any combination of the following:
receiving feedback associated with the multi-dimensional pupillary distance set; and re-training the ML model using the feedback to improve accuracy of pupillary distance measurements;
performing, by the ML model, facial landmark detection for identifying at least one facial landmark associated with the face; and generating a set of pupil center coordinates associated with a pupil center identification;
generating a comprehensive three-dimensional geometrical structure of the face using multi-dimensional analysis of the face; and measuring the first distance, the second distance, and the third distance using the comprehensive three-dimensional geometrical structure;
generating guided instructions for calibrating parameters of the face relative to the image capture device to improve image quality of the plurality of images captured by the image capture device; and presenting the guided instructions via a user interface;
calculating, by a regression model, a final first distance from the first distance vector, a final second distance from the second distance vector, and a final third distance from the third distance vector; and determining, by the regression model, a confidence score associated with each of the final first distance, the final second distance, and the final third distance; and
wherein the ML model is implemented on a server pool comprising a plurality of servers, and the ML model distributes the plurality of images across the plurality of servers for measuring of the first distance, the second distance, and the third distance for each of the plurality of images to improve efficiency of the server pool.

At least a portion of the functionality of the various elements in FIG. 1 can be performed by other elements in FIG. 1, or an entity (e.g., processor 106, web service, server, application program, computing device, etc.) not shown in FIG. 1.

In some examples, the operations illustrated in FIG. 7 can be implemented as software instructions encoded on a computer-readable medium, in hardware programmed or designed to perform the operations, or both. For example, aspects of the disclosure can be implemented as a system on a chip or other circuitry including a plurality of interconnected, electrically conductive elements.

While the aspects of the disclosure have been described in terms of various examples with their associated operations, a person skilled in the art would appreciate that a combination of operations from any number of different examples is also within scope of the aspects of the disclosure.

The term “Wi-Fi” as used herein refers, in some examples, to a wireless local area network using high frequency radio signals for the transmission of data. The term “BLUETOOTH®” as used herein refers, in some examples, to a wireless technology standard for exchanging data over short distances using short wavelength radio transmission. The term “NFC” as used herein refers, in some examples, to a short-range high frequency wireless communication technology for the exchange of data over short distances.

### Example Operating Environment

Although described in connection with an example computing device 102, 220, examples of the disclosure are capable of implementation with numerous other general-purpose or special-purpose computing system environments, configurations, or devices. Examples of well-known computing systems, environments, and/or configurations that may be suitable for use with aspects of the disclosure include, but are not limited to, smart phones, mobile tablets, mobile computing devices, personal computers, server computers, hand-held or laptop devices, multiprocessor systems, gaming consoles, microprocessor-based systems, set top boxes, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, virtual reality (VR) devices, augmented reality (AR) devices, mixed reality devices, holographic device, and the like. Such systems or devices may accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions, or the specific components or modules illustrated in the figures and described herein. Other examples of the disclosure may include different computer-executable instructions or components having more or less functionality than illustrated and described herein. In examples involving a general-purpose computer, aspects of the disclosure transform the general-purpose computer into a special-purpose computing device when configured to execute the instructions described herein.

By way of example and not limitation, computer readable media comprise computer storage media and communication media. Computer storage media include volatile and nonvolatile, removable, and non-removable memory implemented in any method or technology for storage of information such as computer readable instructions, data structures, program modules, and the like. Computer storage media are tangible and mutually exclusive to communication media. Computer storage media are implemented in hardware and exclude carrier waves and propagated signals. Computer storage media for purposes of this disclosure are not signals per se. Example computer storage media include hard disks, flash drives, solid-state memory, phase change random-access memory (PRAM), static random-access memory (SRAM), dynamic random-access memory (DRAM), other types of random-access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transmission medium that may be used to store information for access by a computing device. In contrast, communication media typically embody computer readable instructions, data structures, program modules, or the like in a modulated data signal such as a carrier wave or other transport mechanism and include any information delivery media.

The order of execution or performance of the operations in examples of the disclosure illustrated and described herein is not essential, and may be performed in different sequential manners in various examples. For example, it is contemplated that executing or performing a particular operation before, contemporaneously with, or after another operation is within the scope of aspects of the disclosure. When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

What is claimed is:

1. A method for generating accurate pupillary distances using image data of a remote subject, the method comprising:
receiving a plurality of images of a face of the remote subject from an image capture device;
performing, by a machine learning (ML) model, a pixel-to-millimeter conversion for each image in the plurality of images;
measuring, by the ML model, a set of distances for each image in the plurality of images using a result of the pixel-to-millimeter conversion for each image, each set of distances comprising a first distance between a first pupil and a second pupil of the face, a second distance between the first pupil and a center of the face, and a third distance between the second pupil and the center of the face;
generating a first distance vector representing the first distance for each image of the plurality of images, a second distance vector representing the second distance for each image of the plurality of images, and a third distance vector representing the third distance for each image of the plurality of images; and
generating a multi-dimensional pupillary distance set associated with the first pupil and the second pupil of the remote subject using the first distance vector, the second distance vector, and the third distance vector.

2. The method of claim 1, further comprising:
receiving feedback associated with the multi-dimensional pupillary distance set; and
re-training the ML model using the feedback to improve accuracy of pupillary distance measurements.

3. The method of claim 1, wherein measuring the first distance, the second distance, and the third distance further comprises:
performing, by the ML model, facial landmark detection for identifying at least one facial landmark associated with the face; and
generating a set of pupil center coordinates associated with a pupil center identification.

4. The method of claim 1, further comprising:
generating a comprehensive three-dimensional geometrical structure of the face using multi-dimensional analysis of the face; and
measuring the first distance, the second distance, and the third distance using the comprehensive three-dimensional geometrical structure.

5. The method of claim 1, further comprising;
generating guided instructions for calibrating parameters of the face relative to the image capture device to improve image quality of the plurality of images captured by the image capture device; and
presenting the guided instructions via a user interface.

6. The method of claim 1, further comprising:
calculating, by a regression model in generating the multi-dimensional pupillary distance set, a final first distance from the first distance vector, a final second distance from the second distance vector, and a final third distance from the third distance vector; and
determining, by the regression model, a confidence score associated with each of the final first distance, the final second distance, and the final third distance.

7. The method of claim 1, wherein the ML model is implemented on a server pool comprising a plurality of servers, and the ML model distributes the plurality of images across the plurality of servers for measuring of the first distance, the second distance, and the third distance for each of the plurality of images to improve efficiency of the server pool.

8. A system for generating accurate pupillary distances using image data of a remote subject, the system comprising:
a processor; and
a computer-readable medium storing instructions that are operative upon execution by the processor to:
receive a plurality of images of a face associated with the remote subject from an image capture device;
measure, by a machine learning (ML) model, a set of distances for each image in the plurality of images, each set of distances comprising a first distance between a first pupil and a second pupil of the face, a second distance between the first pupil and a center of the face, and a third distance between the second pupil and the center of the face;
generate a first distance vector representing the first distance for each image of the plurality of images, a second distance vector representing the second distance for each image of the plurality of images, and a third distance vector representing the third distance for each image of the plurality of images; and
generate a multi-dimensional pupillary distance set associated with the first pupil and the second pupil of the remote subject using the first distance vector, the second distance vector, and the third distance vector.

9. The system of claim 8, wherein the computer-readable medium further stores instructions operative by the processor to:
perform, by the ML model, pixel-to-millimeter conversions associated with the plurality of images; and
measure the first distance, the second distance, and the third distance using pixel-to-millimeter conversion results.

10. The system of claim 8, wherein, in measuring the first distance, the second distance, and the third distance, the computer-readable medium further stores instructions operative by the processor to:
perform, by the ML model, facial landmark detection for identifying at least one facial landmark associated with the face; and
generate a set of pupil center coordinates associated with a pupil center identification.

11. The system of claim 8, wherein the computer-readable medium further stores instructions operative by the processor to:
generate a comprehensive three-dimensional geometrical structure of the face using multi-dimensional analysis of the face; and
measure the first distance, the second distance, and the third distance using the comprehensive three-dimensional geometrical structure.

12. The system of claim 8, wherein the computer-readable medium further stores instructions operative by the processor to:
generate guided instructions for calibrating parameters of the face relative to the image capture device to improve image quality of the plurality of images captured by the image capture device; and
present the guided instructions via a user interface.

13. The system of claim 8, wherein the computer-readable medium further stores instructions operative by the processor to:
calculate, by a regression model, a final first distance from the first distance vector, a final second distance from the second distance vector, and a final third distance from the third distance vector; and
determine, by the regression model, a confidence score associated with each of the final first distance, the final second distance, and the final third distance.

14. The system of claim 8, wherein the ML model is implemented on a server pool comprising a plurality of servers, and the ML model distributes the plurality of images across the plurality of servers for measuring of the first distance, second distance, and third distance for each of the plurality of images to improve efficiency of the server pool.

15. A computer-readable medium storing instructions for generating accurate pupillary distances using image data associated with a remote subject, the instructions operative upon execution by a processor to:
receive a plurality of images of a face associated with the remote subject from an image capture device;
measure, by a machine learning (ML) model, a set of distances for each image in the plurality of images, each set of distances comprising a first distance between a first pupil and a second pupil of the face, a second distance between the first pupil and a center of the face, and a third distance between the second pupil and the center of the face;
generate a first distance vector representing the first distance for each image of the plurality of images, a second distance vector representing the second distance for each image of the plurality of images, and a third distance vector representing the third distance for each image of the plurality of images; and
generate a multi-dimensional pupillary distance set associated with the first pupil and the second pupil of the remote subject using the first distance vector, the second distance vector, and the third distance vector.

16. The computer-readable medium of claim 15, wherein the computer-readable medium further stores instructions operative by the processor to:
perform, by the ML model, pixel-to-millimeter conversions associated with the plurality of images; and
measuring the first distance, the second distance, and the third distance using pixel-to-millimeter conversion results.

17. The computer-readable medium of claim 15, wherein, in measuring the first distance, the second distance, and the third distance, the computer-readable medium further stores instructions operative by the processor to:
perform, by the ML model, facial landmark detection for identifying at least one facial landmark associated with the face; and
generate a set of pupil center coordinates associated with a pupil center identification.

18. The computer-readable medium of claim 16, wherein the computer-readable medium further stores instructions operative by the processor to:
generate a comprehensive three-dimensional geometrical structure of the face using multi-dimensional analysis of the face, the comprehensive three-dimensional geometrical structure utilized in measuring the first distance, the second distance, and the third distance.

19. The computer-readable medium of claim 15, wherein the computer-readable medium further stores instructions operative by the processor to:
generate guided instructions for calibrating parameters of the face relative to the image capture device to improve image quality of the plurality of images captured by the image capture device; and
present the guided instructions via a user interface.

20. The computer-readable medium of claim 15, wherein the computer-readable medium further stores instructions operative by the processor to:
calculate, by a regression model, a final first distance from the first distance vector, a final second distance from the second distance vector, and a final third distance from the third distance vector; and
determine, by the regression model, a confidence score associated with each of the final first distance, the final second distance, and the final third distance.
